(19) United States
(12) Patent Application Publication
(10) Pub. No.: US 2025/0332394 A1
(43) Pub. Date: Oct. 30, 2025

(54) Medical Delivery Device

(71) Applicants: Orlance, Inc., Seattle, WA (US); University of Washington, Seattle, WA (US)

(72) Inventors: Hannah Elise Frizzell, Seattle, WA (US); Kristyn Rene Aalto, Kingston, WA (US); Amanda Kay Woodcock, Seattle, WA (US); Dennis Earl McCabe, Seattle, WA (US); Deborah Lynn Fuller, Bainbridge Island, WA (US); James Thomas Fuller, Bainbridge Island, WA (US); Adam Smith, Palm Desert, CA (US)

(21) Appl. No.: 19/262,725

(22) Filed: Jul. 8, 2025

Related U.S. Application Data
(63) Continuation-in-part of application No. 17/571,047, filed on Jan. 7, 2022, now abandoned.
(60) Provisional application No. 63/135,616, filed on Jan. 10, 2021.

(51) Int. Cl. A61M 35/00; A61M 39/22

(52) U.S. Cl. CPC A61M 35/003; A61M 39/22; A61M 2202/0007; A61M 2205/10; A61M 2205/12; A61M 2205/3331; A61M 2205/583; A61M 2205/8218

(57) ABSTRACT

Disclosed and claimed is a medical delivery device with a separable housing comprising a durable component and a disposable component. The durable component comprises a handle portion of the device that may be connected to a compressed gas source to allow for the input of pressurized gas into the device. The disposable component comprises a cartridge containing a plurality of doses of biological or non-biological material and a supersonic barrel through which the dose is propelled out of the device and into the subject. When all of the doses in the cartridge have been administered, or when the device is to be used with a different subject, the cartridge is removed, and a new cartridge attached to the durable component.

### CROSS REFERENCE TO RELATED APPLICATIONS

This application is a continuation-in-part of prior, co-pending U.S. application Ser. No. 17/571,047, filed on Jan. 7, 2022, which claims the benefit of U.S. Provisional Application No. 63/135,616, filed Jan. 10, 2021, which are incorporated by reference herein in their entirety for all purposes.

### GOVERNMENT SUPPORT STATEMENT

This invention was made with government support under Grant Number: R43AI140784 awarded by the National Institutes of Health. The government has certain rights in the invention.

### TECHNICAL FIELD

The subject matter described generally relates to the field of medical devices, and in particular, to a medical delivery device for delivering a vaccine, medication, treatment, or other biological or non-biological material into the epidermis or mucosal tissue of a subject.

### BACKGROUND

Medical delivery devices, such as gene guns, may be used to deliver biological or non-biological material into a subject by accelerating high-density particles to high speeds that allow for epidermal penetration of the material. The use of these devices enables effective delivery of the material while avoiding shortcomings associated with delivery via needle and syringe or jet injectors, including the risk of cross-contamination, accidental needle stick, bruising, or infection. However, conventional gene guns are limited in the maximum dose of gold particles that can be delivered into a single shot to achieve optimum cell viability and in vivo transfection efficiency. Moreover, these devices typically use an internally vented solenoid valve to control the flow of pressurized gas from the gas source to the barrel, resulting in an increased time from solenoid opening to achieve maximum device pressure than if such valve were externally vented. This increased time for solenoid opening on conventional devices reduces the consistency of delivery and penetration of the material between shots.

### BRIEF DESCRIPTION OF THE DRAWINGS

FIG. 1 illustrates a side view of a medical delivery device, according to one embodiment.

FIG. 2 illustrates external components of a housing of the medical delivery device of FIG. 1, according to one embodiment.

FIG. 3 illustrates an internal view of the medical delivery device of FIG. 1, according to one embodiment.

FIG. 4 illustrates a dose counter window of the medical delivery device of FIG. 1, according to one embodiment.

FIG. 5 illustrates a first exploded view of the separable housing of the medical delivery device of FIG. 1, according to one embodiment.

FIG. 6 illustrates a second exploded view of the separable housing of the medical delivery device of FIG. 1, according to one embodiment.

FIG. 7 illustrates the medical delivery device of FIG. 1 connected to a compressed gas source, according to one embodiment.

FIG. 8 illustrates example specifications of the supersonic barrel of the medical delivery device of FIG. 1, according to one embodiment.

FIG. 9 is a flow chart illustrating a method for operating the medical delivery device of FIG. 1, according to one embodiment.

FIG. 10 illustrates perspective, front, and side views of a medical delivery device, according to a second embodiment.

FIG. 11 illustrates an exploded view of a disposable component of the medical delivery device of FIG. 10.

FIGS. 12A-12H illustrate stages of a Geneva wheel advancement mechanism of the medical delivery device of FIG. 10.

FIGS. 13A-13B illustrate top views of the disposable component of the medical delivery device of FIG. 10 with and without the Geneva wheel advancement mechanism.

FIG. 14 illustrates a cross-sectional view of the medical delivery device of FIG. 10.

FIG. 15 illustrates a burst membrane of a dose cartridge chamber of the medical delivery device of FIG. 10.

FIG. 16 is a graph illustrating the performance of burst membrane configurations compared to non-burst membrane medical delivery devices.

FIG. 17 is a graph illustrating the performance of burst membrane configurations operating at a range of burst pressures.

FIG. 18 illustrates a housing of a durable component of the medical delivery device of FIG. 10.

FIG. 19 illustrates an internal center chassis of the durable component of the medical delivery device of FIG. 10.

FIG. 20 illustrates a cassette of the medical delivery device of FIG. 10.

FIG. 21 illustrates installation of the cassette in the disposable component of the medical delivery device of FIG. 10.

FIG. 22 illustrates a disposable attachment method for coupling the disposable component to the durable component of the medical delivery device of FIG. 10.

FIG. 23 illustrates improved clinical utility of the medical delivery device of FIG. 10 over legacy devices.

### DETAILED DESCRIPTION

The Figures (FIGS.) and the following description describe certain embodiments by way of illustration only. One skilled in the art will readily recognize from the following description that alternative embodiments of the structures and methods may be employed without departing from the principles described. Reference will now be made to several embodiments, examples of which are illustrated in the accompanying figures. It is noted that wherever practicable similar or like reference numbers are used in the figures to indicate similar or like functionality.

### Overview and Benefits

Disclosed by way of embodiment is a medical delivery device with a separable housing comprising a durable component and a disposable component. The durable component (referred to throughout as a “reusable body”) comprises a handle portion of the device that may be connected to a compressed gas source to allow for the input of pressurized gas into the device. The disposable component comprises a cartridge containing a plurality of doses of biological or non-biological material and a supersonic barrel through which the dose is propelled out of the device and into the subject. When all of the doses in the cartridge have been administered, or when material is to be delivered to a different subject, the cartridge is removed, and a new cartridge attached to the reusable body. While the primary embodiment discussed herein contemplates a disposable cartridge, in another embodiment, a cylinder containing the doses of material is inserted into a durable cartridge such that the cartridge, in addition to the body, may be used for material delivery to multiple subjects.

The medical delivery device uses a high-velocity stream of gas to accelerate gold particles containing the material from the dose chamber through the supersonic barrel at speeds sufficient to penetrate cells. In one embodiment, the barrel comprises a primary expansion zone beginning at a first distal end of the barrel and an overexpansion zone beginning at an endpoint of a step separating the zones and ending at the second distal end of the barrel in an outlet nozzle that may be placed against the epidermis of a subject (e.g., against the subject's arm or other skin sites) for delivery of the material. Alternatively, the device may be used for mucosal tissue delivery of material into the subject (e.g., into the subject's eyelid, inner cheek, or tongue).

### First Example Device

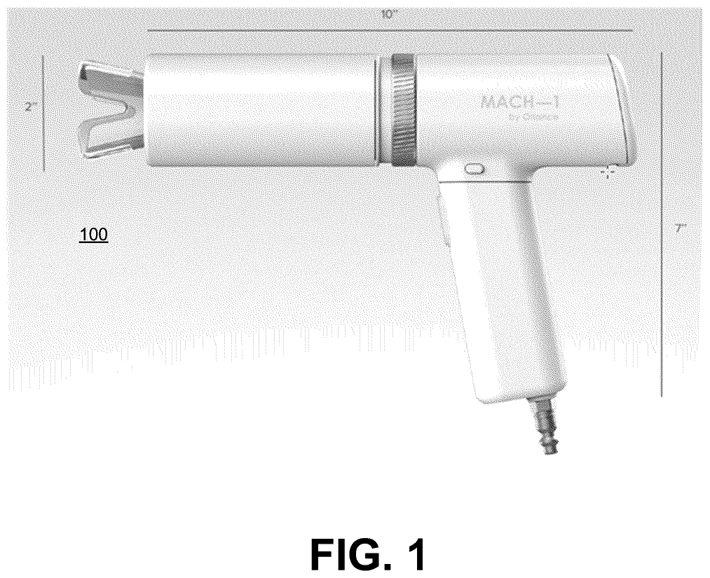

Figure (“FIG.”) 1 illustrates a side view of a medical delivery device 100, according to a first embodiment. The medical delivery device 100 of FIG. 1 may be used to deliver vaccines, such as the COVID-19 vaccine or any other vaccine, medication, treatment, or biological or non-biological therapeutic payload (referred to throughout as “material”) loaded onto gold microparticles into the epidermis or mucosal tissue of a subject, where cells responsible for making retinoic acid reside.

As discussed in more detail below, the device 100 has a separable housing comprising a reusable body on a handle side that is connected to an external compressed gas source and a disposable cartridge side that contains one or more doses of the material. In one embodiment, each cartridge may be used with a single subject (e.g., patient) and contain up to four doses of the material. Accordingly, the cartridge side may be removed (e.g., when the doses have all been administered or for a different subject), and the handle side may be re-used with a different cartridge containing the same or a different type of material for the same or a different subject.

In one embodiment, the medical delivery device 100 has a height of approximately seven inches from the top of the reusable body to the base of the handle and a width of approximately ten inches from a battery at the base of the reusable body to the end of the exterior of the device housing. The device 100 additionally includes a supersonic barrel for delivering the material into the epidermis or mucosal tissue of the subject. As shown in FIG. 1, the terminal end of the barrel extends outwardly from the device housing in a nozzle that may be placed against the subject when the device 100 is to be discharged. However, one of skill in the art will recognize that the device 100 may have a different form factor than in the embodiment described above. For example, in an alternate configuration, the reusable body of the device 100 may be wider and/or have a different shape to accommodate a larger solenoid valve inside the body.

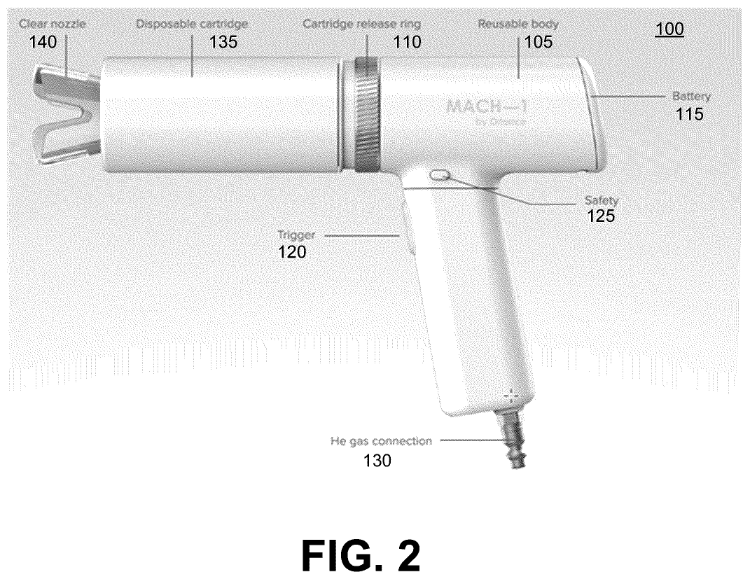

Turning now to FIG. 2, it illustrates external components of the housing of the medical delivery device 100 of FIG. 1, according to one embodiment. In the embodiment illustrated in FIG. 2, the medical delivery device 100 includes a reusable body 105 on a handle side of the device 100 and a disposable cartridge 135 on a barrel side of the device 100. The reusable body 105 includes a cartridge release ring 110, a battery 115, a trigger 120, a safety 125, and a gas connection 130. The disposable cartridge 135 includes a supersonic barrel 230 of which a nozzle portion 140 at the terminal end of the barrel 230 extends outwardly from an interior of the disposable cartridge housing. In other embodiments, the medical delivery device 100 contains different and/or additional elements. In addition, the functions may be distributed among the elements in a different manner than described.

The reusable body 105 may be coupled to the disposable cartridge 135 via the cartridge release ring 110. When the cartridge release ring 110 is engaged, it secures the handle side of the device 100 to the barrel side such that the internal components of the device 100 are operably coupled to allow for operation of the device 100 and the delivery of the material. In one embodiment, the cartridge release ring 110 may be turned, e.g., in a clockwise direction, to secure the cartridge 135 to the body 105 and turned in an opposite, e.g., counterclockwise, direction to decouple the cartridge 135 from the body 105, for example when replacing the disposable cartridge 135. One of skill in the art will recognize that other coupling means for securing the cartridge 135 to the body 105 may be used in other embodiments.

In one embodiment, the body 105 includes a battery 115 that powers the electrical system of the device 100, enabling the device 100 to be discharged when the trigger 120 is depressed and the safety 125 disengaged. The battery 115 may be removable, for example, to allow the battery to be replaced or charged via a separate charging mechanism. While a majority of the battery length may be positioned in a chamber inside the housing at the base of the body 105, a portion of the battery 115 may be positioned on an outside of the housing to allow a user (e.g., a clinician) to easily remove the battery 115 from the body 105. Once charged, the battery 115 may be reinserted into the chamber. In another embodiment, the battery 115 may be charged while engaged with the body 105, e.g., via a charging cable or other mechanism. While the battery 115 is shown as located at a base of the body 105, one of skill in the art will recognize that the battery could be positioned elsewhere on the device 100, such as in the handle.

The trigger 120 is located on a handle portion of the body 105 and controls the flow of pressurized gas into the device 100, causing activation of the pressure delivery system inside the housing when the trigger 120 is depressed. In one embodiment, the trigger is electrical and is driven by the battery 115, as discussed above. Alternatively, the trigger is mechanical and powered directly from wall power.

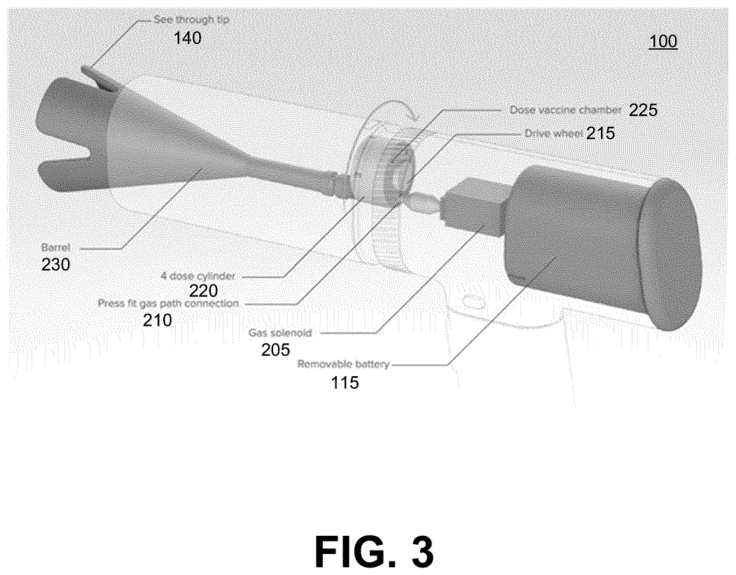

When activated, the trigger 120 causes a solenoid valve (shown and discussed below with respect to FIG. 3) to open, allowing input of pressurized gas via the gas connection 130 into the dose chamber. In one embodiment, the trigger 120 must be activated for at least ten milliseconds (ms) to achieve sufficient particle penetration and delivery pressure to release particles from the cartridge 135.

The trigger 120 is functional only when the safety 125 is disengaged. In one embodiment, the safety 125 is a button located at a top of the handle portion and is disengaged when pushed in. Once depressed, the safety 125 activates the trigger 120 for a specified period of time, e.g., ten seconds, thirty seconds, etc. If the device 100 is not discharged (i.e., the trigger 120 not depressed) within the specified time, the safety 125 is reengaged such that the user must press the safety 125 again to reactivate the trigger 120. In one embodiment, after the device 100 is discharged, there may be a delay (e.g., of N seconds) before the device 100 may be fired again.

The gas connection 130 is an inlet at the base of the handle that enables the device 100 to be connected to an external compressed gas source via a hose. As discussed below with respect to FIG. 3, activation of the trigger causes the input of pressurized gas obtained via the gas connection 130 through the solenoid valve and gas path connection into the dose chamber to allow the material in the chamber to be propelled through the barrel into the epidermis or mucosal tissue of the subject. In various embodiments, helium, nitrogen, or hydrogen gas may be used, however one of skill in the art will recognize that other pressurized gasses may be used in other embodiments. Additionally, in one embodiment, the gas tank is pressurized to approximately 1500 pounds per square inch (PSI). Alternatively, the gas tank is pressurized to above 500 PSI for 400 PSI delivery of the dose or to above 300 PSI for 200 PSI delivery.

The cartridge 135 may be coupled to the reusable body 105 via the cartridge release ring 110 and contain one or more doses of the material. As discussed below with respect to FIG. 3, an interior of the cartridge 135 includes a dose cylinder having a plurality of chambers, each configured to carry a dose of material for delivery to a subject. The cylinder is coupled to a first distal end of the elongated barrel 230 that spans the length of the cartridge 135 and protrudes outwardly from an opening in the cartridge. As shown in FIG. 2, the second distal end of the barrel comprises a nozzle 140 that may be placed against the subject (e.g., against the subject's arm or other skin sites) for delivery of the material into the epidermis. Alternatively, the device 100 may be used for mucosal tissue delivery of the material. The configuration of the nozzle 140 provides sufficient surface area to enable material penetration into the epidermis or mucosal tissue of the subject. Additionally, while the nozzle 140 shown in FIG. 2 is transparent, in other embodiments, the nozzle 140 is opaque.

FIG. 3 illustrates an internal view of the medical delivery device 100 of FIG. 1, according to one embodiment. In the embodiment shown in FIG. 3, the internal components of the medical delivery device 100 include the removable battery 115, a solenoid valve 205, a gas path connection 210, a drive wheel 215, a dose cylinder 220, a dose chamber 225, and a barrel 230.

The solenoid valve 205 opens and closes to control the flow of pressurized gas into the dose chamber. In a default state (i.e., when the trigger 120 is not depressed and/or the safety 125 is engaged), the solenoid valve 205 is closed such that pressurized gas does not enter the chamber 225 containing the dose of material. Activation of the trigger 120 and disengagement of the safety 125 activates the solenoid valve (i.e., causes the solenoid valve 205 to open) and permits the gas to enter the cartridge through the opening in the valve 205. The solenoid valve 205 remains open when the trigger 120 is depressed.

The solenoid valve 205 may be internally vented or externally vented. In one embodiment, use of an externally vented solenoid valve 205 lowers the rise time (i.e., the time from the opening of the solenoid valve 205 to achieve maximum pressure) as compared to a conventional internally vented valve. High-pressure gas flowing through the solenoid valve 205 causes the gold particles in the dose chamber 225 to become dislodged and begin to flow through the barrel 230. Accordingly, the rapid increase in pressure achieved with an externally vented solenoid valve 205 allows for optimal acceleration of the gold particles.

In an alternate configuration, a burst membrane is used with an internally vented solenoid valve 205 to control the flow of gas into the dose chamber 225. The burst membrane may be comprised of gas-impermeable aluminized mylar such that gas cannot pass into the chamber 225 until a pressure threshold is exceeded and the membrane has burst. The burst membrane is discussed in more detail below in connection with FIGS. 15-17.

In various embodiments, the device 100 is operated under conditions ranging from 200-500 PSI. In a configuration in which approximately 400 PSI of supplied pressure is used, the device 100 delivers high-pressure gas flow with an average rise time of 2.30±0.08 ms to an average peak pressure of 309.44±5.98 PSI to enter the dose chamber 225. Such a pressure delivery profile allows for release of the material from the chamber 225 under high pressure conditions to achieve the required particle acceleration speeds for epidermal or mucosal tissue delivery and penetration. Upon release of the trigger 120, the solenoid valve 205 closes, and the pressure downstream of the valve 205 drops back down to 0 PSI. In one embodiment, maximum pressure is achieved when the solenoid valve 205 remains open for a time period greater than or equal to the rise time of approximately 2 ms.

Additionally, while the device 100 is standardly operated under conditions of an input pressure of 400 PSI, in other embodiments, the device 100 uses an operating pressure of 200 PSI due to enhanced particle acceleration resulting from the supersonic barrel 230, achieving a full particle release and delivery profile compared to 400 PSI. Operation of the device 100 at an input pressure of 200 PSI reduces the noise generated by the device 100 and provides compatibility with solenoid valves having different sizes and weights as compared to operation at a 400 PSI input pressure. In embodiments in which the input pressure is 200 PSI, the outlet pressure of the solenoid valve 205 is approximately 164.75±4.04 PSI with a rise time of approximately 2.29±0.23 ms. Additionally, in various embodiments, valves having varying opening mechanisms (direct or indirect), flow coefficients, and weights are used.

The gas path connection 210 is a chamber connecting the solenoid valve 205 to the dose chamber 225. When the solenoid valve 205 is open, the pressurized gas flows through the gas path connection 210 into the chamber 225.

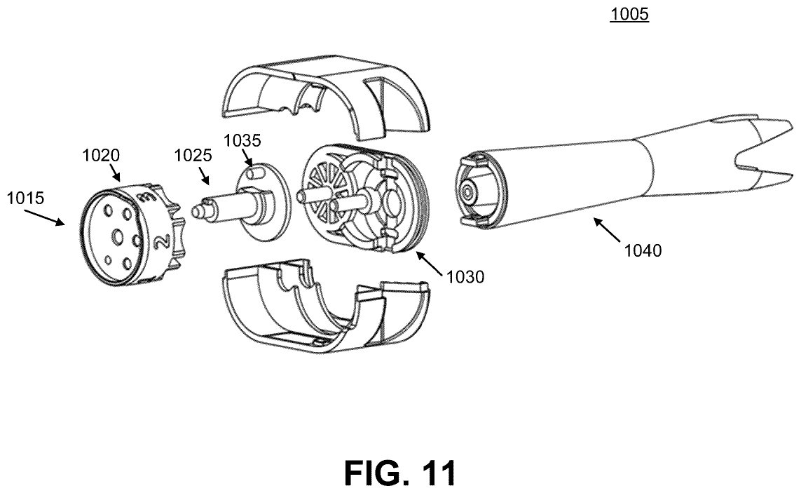

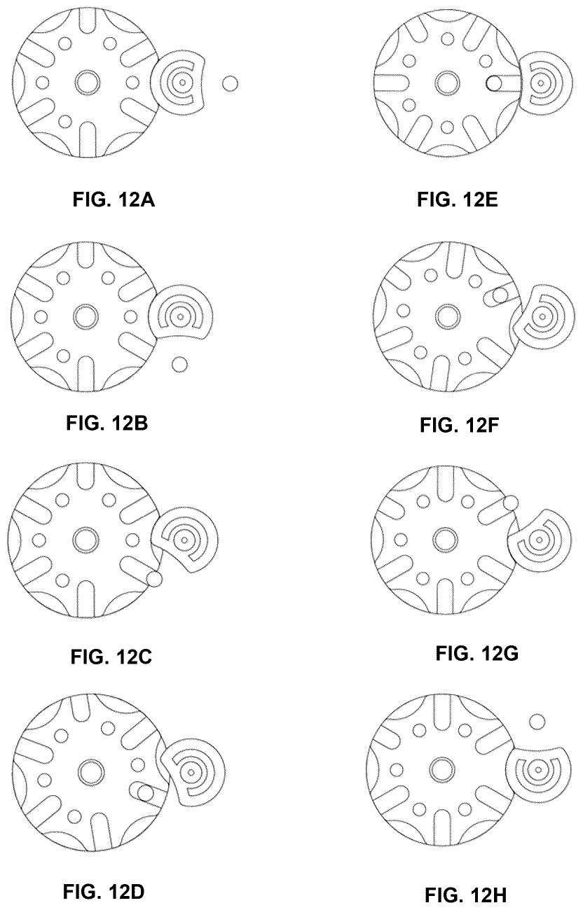

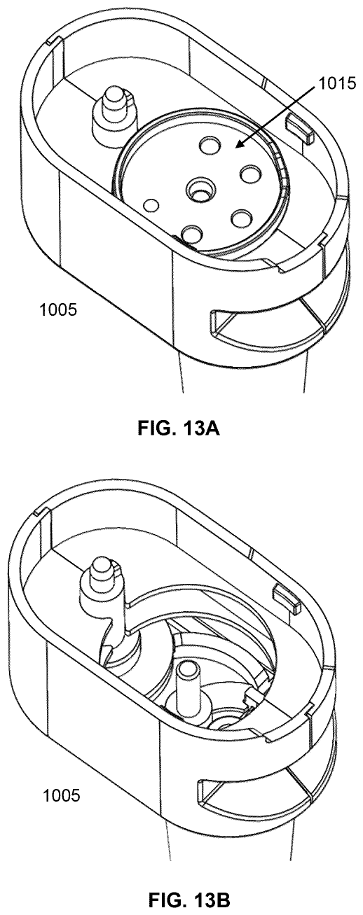

The drive wheel 215 is a chamber advancement mechanism on the handle-side of the device 100 that causes the dose cylinder 225 containing the material at the barrel-side to rotate after each dose is administered. In one embodiment, advancement of the cylinder 225 is automatic and not user-dependent. Operation of the drive wheel 215 is discussed below with respect to FIGS. 11 and 12A-12H.

The dose cylinder 220 is located in the disposable cartridge 135 on the barrel-side of the device 100 adjacent to the drive wheel 215 inside the housing of the reusable body 105 on the handle-side. The dose cylinder 220 comprises a plurality of dose chambers 225 that each contain a single dose of the material. While the embodiment shown in FIG. 3 and described herein contemplates a four-dose cylinder, one of skill in the art will recognize that the cylinder 220 may contain additional or fewer chambers in other embodiments to enable delivery of different numbers of material doses. As discussed above and below, the drive wheel 215 causes the cylinder 220 to rotate to advance to a first dose chamber 225, to each subsequent chamber 225 after discharge, and to a hard stop after the final dose has been administered. As shown in FIG. 3, each chamber 225 is labeled with a corresponding dose number.

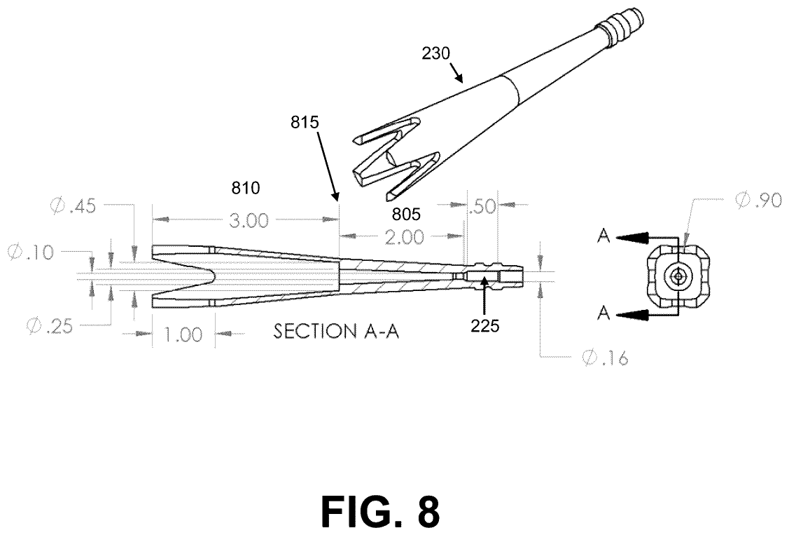

The barrel 230 (also referred to as a “supersonic barrel”) is positioned inside the disposable cartridge 135 and has an elongated body that extends from a first distal end where the barrel 230 is coupled to the cylinder 220 to a second distal end at an outlet of the device 100. The second distal end of the barrel 230 may be placed flush against the epidermis or mucosal tissue of the subject. The barrel 230 is shaped to allow particles from the dose chamber 225 and propelled by the pressurized gas to achieve at least a target velocity at the second distal end (i.e., for penetration into the epidermis or mucosal tissue). In one embodiment, the barrel 230 includes a primary expansion zone beginning at the first distal end and an overexpansion zone having a conical shape beginning at an approximate midpoint of the barrel 230 and expanding in diameter to the second distal end. Example specifications of the supersonic barrel are shown and discussed below with respect to FIG. 8.

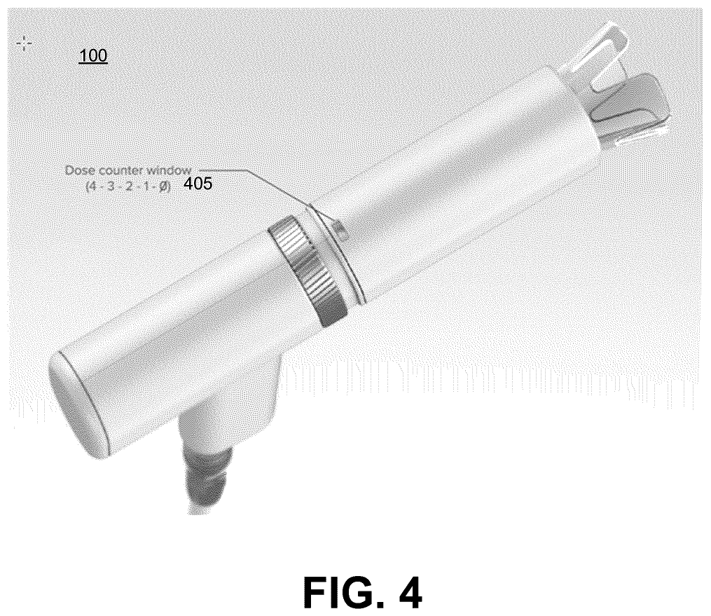

FIG. 4 illustrates a dose counter window 405 of the medical delivery device 100 of FIG. 1, according to one embodiment. In one embodiment, the window 405 comprises a cut-out in the housing of the disposable cartridge 135 such that the dose number located on the outside of each dose chamber 225 is viewable to the user (e.g., the clinician administering the dose), indicating a number of remaining doses of material available in the disposable cartridge 135. After each dose is administered and the cylinder 220 rotated to a subsequent dose chamber 225, the window 405 displays the updated number of available doses. Once the final dose has been administered, the counter window indicates that no remaining doses are available, such that the cartridge 135 may be discarded and replaced with a new cartridge 135 containing the same or different material.

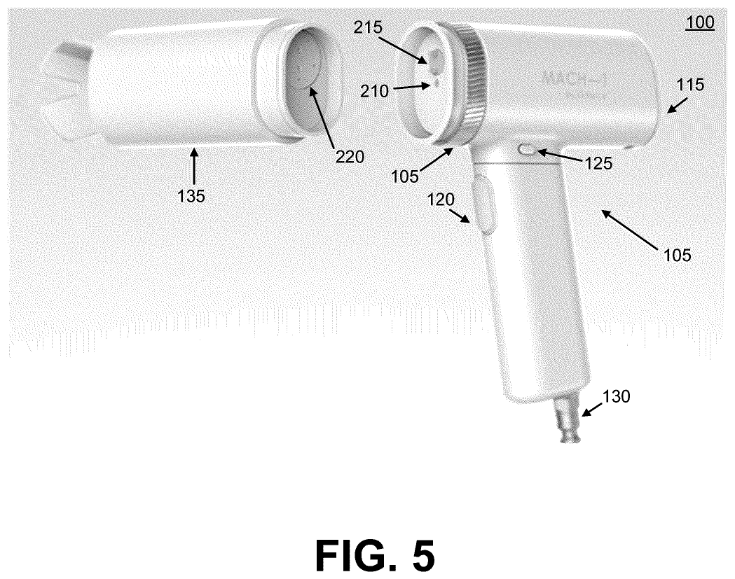

FIG. 5 illustrates a first exploded view of the separable housing of the medical delivery device 100 of FIG. 1, according to one embodiment. As shown in FIG. 5 and discussed above, the housing of the device 100 is separable into two portions for the replacement of the dose cartridge 135. Components located on the reusable body 105 include the cartridge release ring 105, battery 115, trigger 120, safety 125, gas connection 130, and drive wheel 215, which causes the dose cylinder 220 in the disposable cartridge 135 to turn after each dose is administered. Located below the drive wheel 215 in FIG. 5 is the outlet of the gas path connection 210. When the device 110 is assembled (i.e., the disposable cartridge 135 coupled to the reusable body 105 via the cartridge release ring 110), the outlet of the gas path connection 210 is positioned flush against a dose chamber 225 in the cylinder 220. Accordingly, in the embodiment shown in FIG. 5, a dose chamber 225 in the discharge position is the chamber 225 located at the bottom of the dose cylinder 220, and a dose of the material may be discharged from the cylinder 220 when the chamber 225 in which the dose is contained is rotated to the bottom of the cylinder 220. One of skill in the art will recognize that, in other configurations, the gas path connection 210 may be positioned elsewhere, such as above the drive wheel 215.

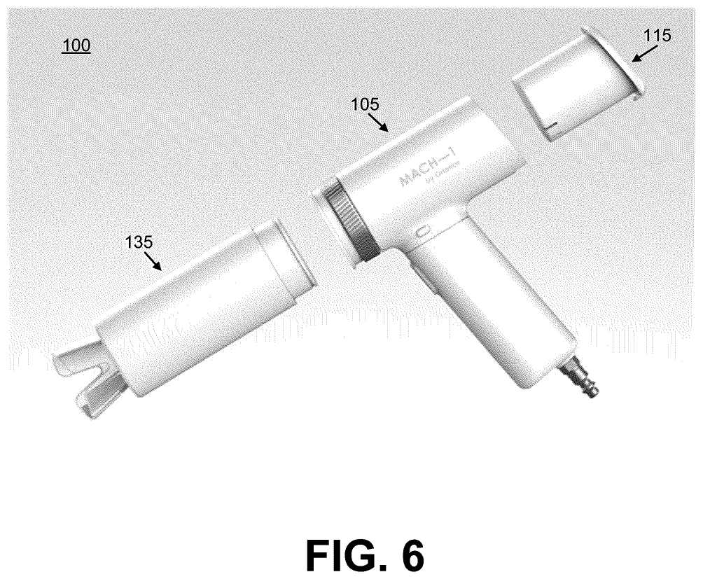

FIG. 6 illustrates a second exploded view of the separable housing of the medical delivery device 100 of FIG. 1, according to one embodiment. As shown in FIG. 6 and discussed above, the battery 115 may be removed from the device 100 for charging or replacement. In some embodiments, the trigger 120 is electrical and is driven by the battery 115. However, in other embodiments, the trigger 120 is mechanical and does not require power.

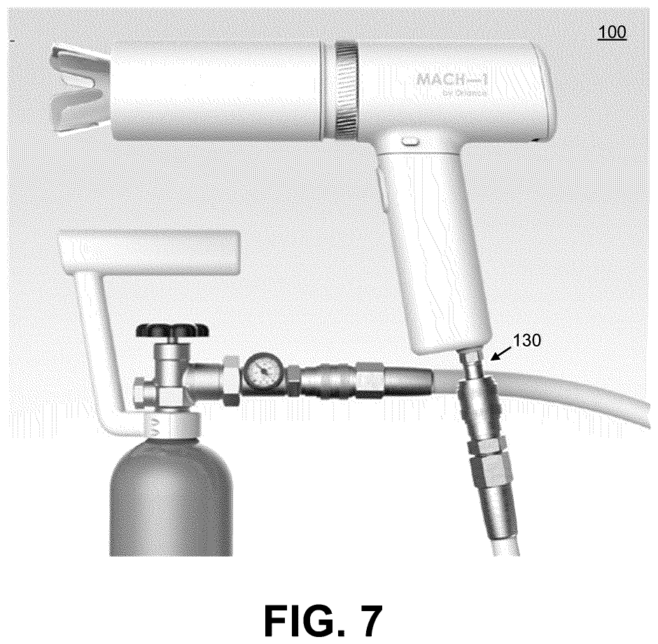

FIG. 7 illustrates the medical delivery device 100 of FIG. 1 connected to a compressed gas source, according to one embodiment. As shown in FIG. 7 and discussed above, the device 100 is connected to the gas source via a hose attached at a first end to the gas source and at a second end to the gas connection 130 on the device 100. In one embodiment, activation of the trigger 120 causes the input of pressurized gas obtained via the gas connection 130 through the solenoid valve 205 and gas path connection 210 into the dose chamber 225 to allow the material to be propelled through the barrel 230 into the epidermis or mucosal tissue of the subject

FIG. 8 illustrates example specifications of the supersonic barrel 230 of the medical delivery device 100 of FIG. 1, according to one embodiment. A target gas velocity through the barrel 230 is a supersonic velocity. Use of a supersonic barrel, such as the barrel 230, optimizes the density of gold delivered throughout the target to maximize intracellular delivery of particles into more cells while retaining high cell viability, with higher maximum particle deposition at input pressures of 200-500 PSI. The supersonic barrel 230 also improves gold particle penetration compared to legacy barrels.

As discussed above with respect to FIG. 3, the barrel 230 has an elongated body that extends from a first distal end where the barrel 230 is coupled to the cylinder 220 to a second distal end at an outlet of the device 100 that is placed against the subject for delivery of the material into the epidermis or mucosal tissue. The barrel 230 includes a primary expansion zone 805 beginning at the first distal end and an overexpansion zone 810 beginning at an approximate midpoint of the barrel 230. In one embodiment, the primary expansion zone 805 has a first inner diameter of approximately 0.08-0.12 inches at the first distal end, a second inner diameter of approximately 0.16-0.25 inches, and a length of approximately 1.6-3.0 inches. The overexpansion zone 810 has a first inner diameter of approximately 0.16-0.24 inches, a second inner diameter of approximately 0.56-0.84 inches, and a length of approximately 1.6-3.0 inches. In one example configuration, the second inner diameter of the primary expansion zone 805 is 0.25 inches, and the second inner diameter of the overexpansion zone is 0.75 inches.

In one embodiment, the overexpansion zone 810 has an inner-diameter expansion-to-length ratio that is higher than the inner-diameter expansion to length ratio of the primary expansion zone 805. For example, the overexpansion zone 810 inner-diameter expansion-to-length ratio may be twice or at least 1.5 times as high as the inner-diameter expansion to length ratio of the primary expansion zone 805.

The primary expansion zone 805 and the overexpansion zone 810 are separated by a step 815 that breaks the high velocity jet away from the wall of the barrel 230 in a clean fashion. In one embodiment, the step 815 is approximately 0.1 inches in length radially such that, when the final diameter of the primary expansion zone 805 is 0.25 inches, the diameter at the step 815 is 0.35 inches (0.25 inches at the terminal end of the primary expansion zone 805 plus 0.1 inches radial step). In this embodiment, the final diameter at the terminal end of the overexpansion zone 810 is 0.75 inches. The step 815 may have a constant diameter over its length and comprise an orifice that enables the downstream flow of particles to be free of boundary effects or conditions and allow the flow to be supersonic and separated from the inner wall of the barrel 230.

The dimensions of the supersonic barrel 230 discussed above are for example only. In alternate embodiments, the dimensions and ratios may be within 10-100% of the numbers listed above. The dimensions may also be proportionally scaled in various embodiments.

### Example Method

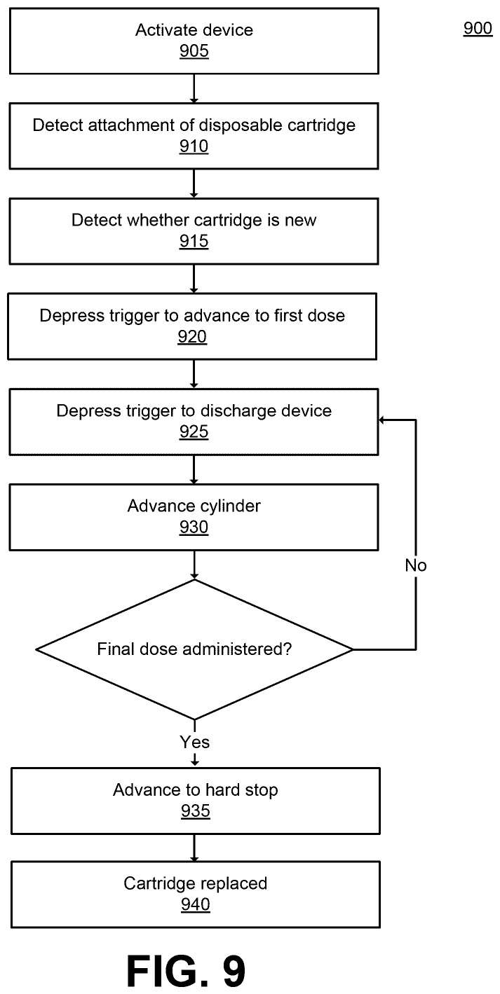

FIG. 9 is a flow chart illustrating a method 900 for operating the medical delivery device 100 of FIG. 1, according to one embodiment. In some embodiments, the operations in the method 900 are performed in a different order or can include different or additional steps.

In the embodiment shown in FIG. 9, the method 900 begins by activating 905 the device 100 (e.g., powering on the device 100 via an “on/off” switch or similar activation mechanism). At 910, a reed switch is used to detect whether a disposable cartridge 135 containing one or more doses of material is coupled to the reusable body 105. The device 100 cannot be discharged if no cartridge 135 is detected.

At 915, absolute position detection is used to verify the device position and detect whether a cartridge is “new.” As discussed above, each cartridge 135 is used with a single subject (e.g., patient), such that the cartridge 135 must be replaced if the device 100 is to be used to administer material to a different patient.

The trigger 120 is depressed 920 a first time to “purge” the device 100, causing the drive wheel 215 to advance the dose cylinder 220 to a first chamber 225 containing a dose of material. In embodiments in which the cartridge 135 contains four chambers 225 containing four doses of the material, the trigger 120 is depressed 925 four additional times to discharge the doses into the epidermis or mucosal tissue

After each discharge of the device 100, the drive wheel 215 advances 930 the dose cylinder 220 to a subsequent chamber 225. After the final dose is administered, the drive wheel 215 advances 935 to a hard stop that prevents the device 100 from firing, and the cartridge 135 is replaced 940.

### Second Example Device

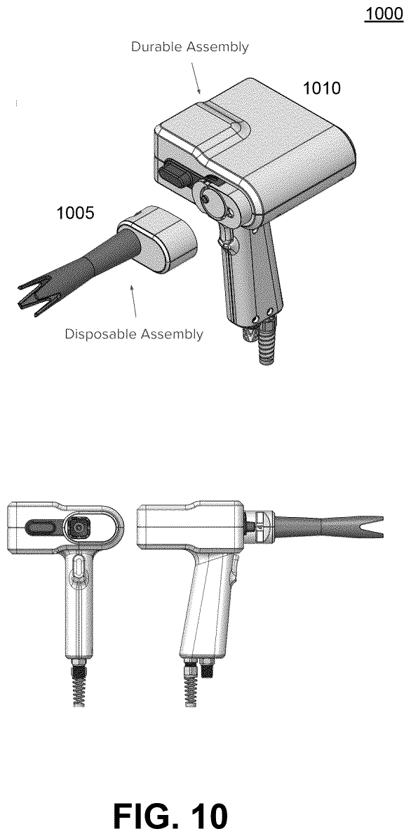

FIG. 10 illustrates perspective, front, and side views of a medical delivery device 1000, according to a second embodiment. Like the device 100, the device 1000 comprises a disposable component 1005 (also referred to as a disposable assembly) and a durable component 1010 (also referred to as a durable assembly). The device 1000 includes a lightweight housing (e.g., comprised of plastic) to ensure maximum maneuverability of the device. A cartridge advancement mechanism within the housing uses a Geneva wheel to ensure precise alignment of the cartridge to the flow path during firing and to automatically align a next dose to the flow path after a dose administration. In one embodiment, a DC solenoid (e.g., 12V or 24V) is used, reducing electrical safety requirements compared to an AC (internally vented) solenoid and yielding equivalent performance. Use of a DC solenoid also allows clinical development and use with a battery-operated device for usability, portability, and compatibility with clinical settings considering EMF, electrical insulation in a multi-device suite, and patient safety issues. A plurality of sensors are implemented into the device 1000 to detect dose cartridge status. For example, a sensor sensing the disposable attachment prevents the solenoid from firing if the disposable component 1005 is not in the start position, preventing accidental cross-contamination between patients (e.g., by accidental re-use of a partially or previously-used disposable component). An end sensor and mechanical hard stop prevent the solenoid from firing if the cartridge has been fully delivered, and attachment sensors prevent the solenoid from firing if the disposable component 1005 is not securely coupled to the durable component 1010. Moreover, a delayed delivery trigger prevents the device 1000 from firing until the trigger has been depressed for at least a threshold period of time (e.g., a half second) to prevent accidental firing.

The device 1000 includes an LED indicator to indicate to the operator the device status (e.g., power, ready to fire, error states). When an error state is displayed, the device function is disabled. A printed circuit board assembly (PCBA) controls solenoid timing, button hold timing, motor torque, and speed. Finally, a disposable clamping mechanism ensures a consistently aligned and secured gas path between the disposable and durable components. Additional details of the second medical delivery device 1000 are provided below with respect to FIGS. 11-22.

FIG. 11 illustrates an exploded view of a disposable component 1005 of the medical delivery device 1000, according to one embodiment. In the displayed embodiment, the disposable component 1005 comprises a housing top, a housing bottom, a housing front, a drive shaft, and a supersonic nozzle body 1040. A cassette 1015 containing a plurality of dose chambers and an external dose indicator 1020 interfaces with the disposable component 1005. In one embodiment, a Geneva wheel mechanism is used for advancing the cassette 1015 to deliver doses of the biological or non-biological material to a subject. A Geneva drive shaft 1025 interacts with the cassette 1015 (Geneva wheel) to advance and precisely lock the cassette 1015 into position. The cassette 1015 and the Geneva drive shaft 1025 rotate around a center post 1030 within the disposable component 1005, allowing the components to interact smoothly during operation. The outer diameter of the Geneva drive shaft 1025 locks into the cassette 1015 fixedly aligning the gas path within the disposable component 1005. As the

Geneva drive shaft 1025 rotates the clearance portion of the outer diameter of the Geneva drive shaft 1025 allows the cassette 1015 to rotate as an actuator pin 1035 on the Geneva drive shaft 1025 advances the cassette 1015 into the next position to ensure that each dose is aligned correctly for delivery. After the movement, the cassette 1015 is locked back into position, maintaining alignment and accuracy of the gas path. A large rotation range (approximately 180 degrees) of the Geneva drive shaft 1025 maintains a fixed alignment of the gas path within the disposable component 1005.

In one embodiment, a motor drive shaft position sensor with the durable component 1010 of the device 1000 stops the motor drive shaft and the interconnected Geneva drive shaft 1025 within the rotation range of the Geneva drive shaft 1025 to maintain fixed alignment of the gas path within the disposable component 1005. Use of the Geneva mechanism on the disposable component 1005 ensures that the disposable gas path aligns properly, locks securely, and advances the dose indicator with precision.

FIGS. 12A-12H illustrate stages of the Geneva wheel advancement mechanism of the medical delivery device 1000. As illustrated in FIGS. 12A-12H, the Geneva mechanism components include the cassette 1015 having a plurality of dose chambers and the Geneva drive shaft 1025 having an actuator pin 1035. The cassette 1015 also includes a plurality of slots into which the pin rotates during rotation and advancement of the mechanism.

For example, FIG. 12A illustrates a first stage of the mechanism in which the actuator pin 1035 is positioned at 3 o'clock. In the first stage, the actuator pin 1035 is fully locked such that the cassette 1015 (Geneva wheel) and the dose chamber are securely aligned with the gas path and supersonic nozzle body 1040. FIG. 12B illustrates a second stage in which the actuator pin 1035 has rotated to a 6 o'clock position in response to motion by the Geneva drive shaft 1025 and remains fully locked and aligned with the delivery path.

FIG. 12C illustrates a third stage of the mechanism in which rotation of the Geneva drive shaft 1025 has advanced the actuator pin 1035 to a 7 o'clock position in which the cassette 1015 is minimally locked and about to advance. The locking engagement between the Geneva drive shaft 1025 and cassette 1015 begins to loosen slightly, creating clearance for movement, and the actuator pin 1035 begins to push on the next slot in the cassette 1015, initiating its rotation to advance to the next dose chamber alignment. While minimally locked, the cassette 1015 is still controlled to ensure that rotation occurs precisely without misalignment.

FIG. 12D illustrates a fourth stage of the mechanism in which the actuator pin 1035 has rotated to an 8 o'clock position. With the pin 1035 in the 8 o'clock position, the mechanism is unlocked such that the cassette 1015 rotates under the force exerted by the pin 1035. The pin 1035 pushes the cassette 1015, initiating its movement toward the next dose chamber position. FIG. 12E illustrates a fifth stage of the mechanism in which the actuator pin 1035 is positioned at 9 o'clock and is approximately halfway through the advancement process. The cassette 1015 is still in motion, with the slot guiding the pin 1035. In this position, the cassette 1015 is partially rotated and the next dose chamber is nearing alignment with the gas path.

FIG. 12F illustrates a sixth stage of the mechanism in which the actuator pin 1035 has rotated to a 10 o'clock position. The mechanism remains unlocked and the pin 1035 continues rotate on the Geneva drive shaft 1025. In this stage, the mechanism is advancing with the dose chamber almost aligned with the gas path and the pin 1035 pushing the cassette 1015 closer to the locked state.

FIG. 12G illustrates a seventh stage of the mechanism in which the mechanism is minimally locked and about to end advancement with the pin at an 11 o'clock position. The rotation of the cassette 1015 is about to stop as the actuator pin 1035 reaches the edge of the slot, slowing rotation and preparing the cassette 1015 to lock securely into position. The minimally locked state ensures that the alignment is refined and the dose chamber is in its final position.

Finally, FIG. 12H illustrates an eighth stage of the mechanism in which the actuator pin 1035 is rotated to a 12 o'clock position and the mechanism is in a fully locked state. The dose chamber is locked into place such that the dose is ready for delivery via the supersonic nozzle body 1040. The outer diameter of the Geneva drive shaft 1025 fully engages with the cassette 1015 to ensure that no additional movement occurs until a next cycle begins. In one embodiment, a final slot of the cassette 1015 is shorter than the preceding slots, creating a final lockout position beyond which the Geneva wheel cannot advance, preventing re-use of the cassette 1015.

FIGS. 13A-13B illustrate top views of the disposable component 1005 of the device 1000 with and without the Geneva wheel advancement mechanism. In FIG. 13A, the cassette 1015 is illustrated in position in the disposable component 1005 and mechanically coupled to the Geneva drive shaft 1025 (not shown). As illustrated in FIG. 13B, the bottom of the cassette 1015 is positioned on top of two planar surfaces within the disposable component 1005 to ensure that the cassette 1015 remains stable during operation and does not exert downward pressure on the Geneva drive shaft 1025. When the gas pressure builds within the disposable component 1005 during delivery, the pressure forces the cassette 1015 upward against the face of the supersonic nozzle body 1040. The pressure maintains the alignment of the cassette 1015 with the nozzle, ensuring a proper seal and efficient gas path delivery. Because the cassette 1015 is supported by the flat surfaces, the Geneva drive shaft 1025 is not compressed or clamped by the cassette 1015 and can rotate without obstruction regardless of the movement of the cassette 1015 or alignment with the supersonic nozzle body 1040.

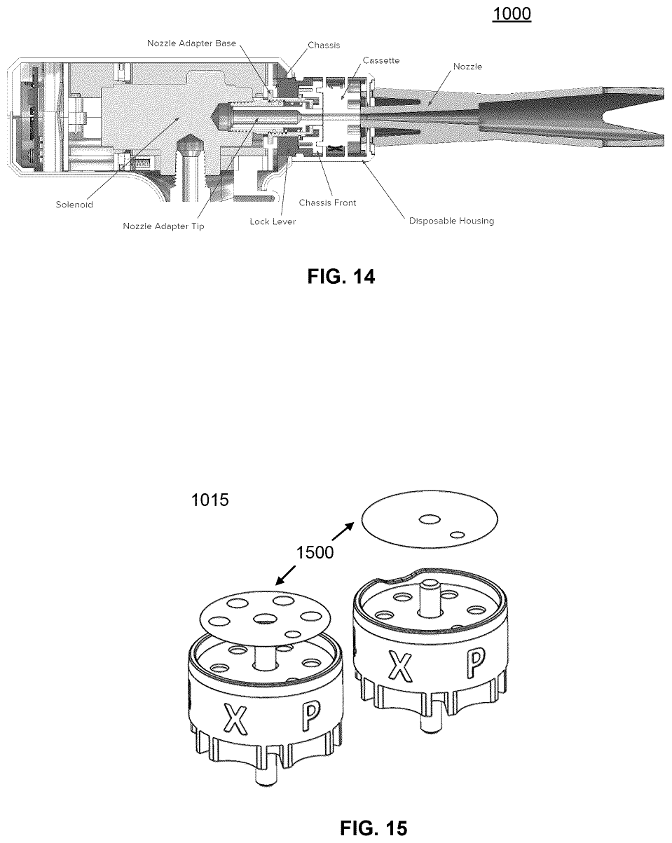

FIG. 14 illustrates a cross sectional view of the medical delivery device 1000 in which the disposable component 1005 is securely coupled to the durable component 1010. In the illustrated embodiment, the gas path alignment between the durable component 1010 and the disposable component 1005 is achieved by precisely fitting the disposable housing onto the chassis front within the durable component 1010. The gas solenoid valve is located within the chassis of the durable component 1010. The position of the valve is controlled using a National Pipe Thread (NPT) nozzle adapter threaded into a NPT exhaust port of the solenoid valve. The use of the threaded NPT interface ensures tight sealing and precise positioning of the solenoid valve within the durable component 1010. The NPT nozzle adapter assembly is precisely located within the chassis assembly of the durable component 1010. In one embodiment, the elevated ring on the top face of the cassette 1015 is a tapered ring that concentrically aligns to a circular tapered groove in the chassis front of the durable component 1010. This concentric alignment of the NPT nozzle adapter, chassis front, and cassette 1015 ensures that the gas path originating from the solenoid valve flows accurately to the nozzle body.

The NPT nozzle adapter assembly is comprised of the threaded component that mounts into the NPT exhaust port of the solenoid valve as well as a front cap and the moveable nozzle adapter. In one embodiment, the moveable nozzle adapter is spring loaded against the cassette 1015 when the disposable component 1005 is mounted on the durable component 1010. The NPT nozzle adapter assembly is further concentrically aligned to facilitate alignment of the durable gas path to the disposable gas path.

FIG. 15 illustrates a burst membrane 1500 of the cassette 1015 of the medical delivery device 1000. In one embodiment, each dose cartridge chamber in the cassette 1015 is protected by an assembly of adhesive and thin plastic film (the burst membrane 1500), which protect the chamber contents from contamination and external exposure until the point of delivery. The adhesive does not cover the chamber, which allows the film (membrane 1500) to burst under targeted gas pressure. Specifically, the membrane 1500 covers the chambers until a gas pressure is great enough to deform the film into the chamber, causing the film covering the chamber in the discharge position to burst and the material contained in that chamber to be expelled through the gas path.

The other (unused) chambers remain sealed by the film, ensuring sterility and preserving the contents until their respective alignment and activation by gas pressure. The selective bursting mechanism of the burst membrane 1500 ensures that only the desired dose chamber (i.e., the chamber in the discharge position) is accessed at any given point, maintaining precision in sequential dosing. Moreover, bursting of the top layer film may also cause the gas pressure and time profile to be sharper or more ideal for dose. In some embodiments, use of the burst membrane increases the pressure wave interacting with the dose and increases particle acceleration by acting as an externally vented solenoid valve. Inclusion of the burst membrane further enables use of an internal venting solenoid with the performance of a modified external vented solenoid as the burst membrane protects doses from being exposed to low pressure that is vented through the system with the internal venting solenoid.

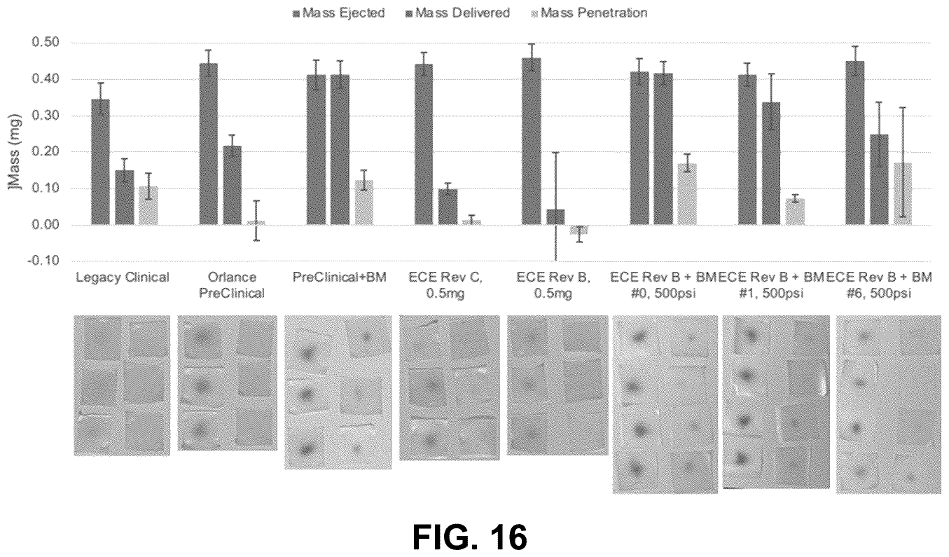

FIG. 16 is a graph illustrating the performance of burst membranes configurations compared to non-burst membrane medical delivery devices. In different embodiments, the device 1000 uses burst membranes 1500 having varying thicknesses and burst pressures. For example, a first (“thick”) burst membrane 1500 may be comprised of high-temperature polyester film, have a thickness of 0.001 inches, and operate at a burst pressure between 350 and 400 PSI; a second (“medium”) burst membrane 1500 may be comprised of a clear polyester film, have a thickness of 0.0005 inches, and operate at a burst pressure of between 250 to 300 PSI; and a third (“thin”) burst membrane 1500 may be comprised of mylar, have a thickness of 5 microns, and operate at a burst pressure of between 120 and 140 PSI. One of skill in the art will recognize that other materials having a range of thicknesses and burst pressures may be used in other embodiments. Moreover, in different embodiments, varying device driving pressures (e.g., ranging from approximately 200-500 PSI) may be used.

As illustrated in FIG. 16, the device 1000 (represented as “ECE Rev B” and “ECE Rev C” in the chart) was tested with three different burst membranes 1500 (“BME #0,” “BME #1,” and “BME #6”) at a driving pressure of 500 PSI and measured against legacy delivery devices (“Legacy Clinical,” “Orlance PreClinical,” and “PreClinical+BM”). The graph of FIG. 16 illustrates that the configurations having a burst membrane 1500 improve mass delivery and penetration compared to the legacy devices.

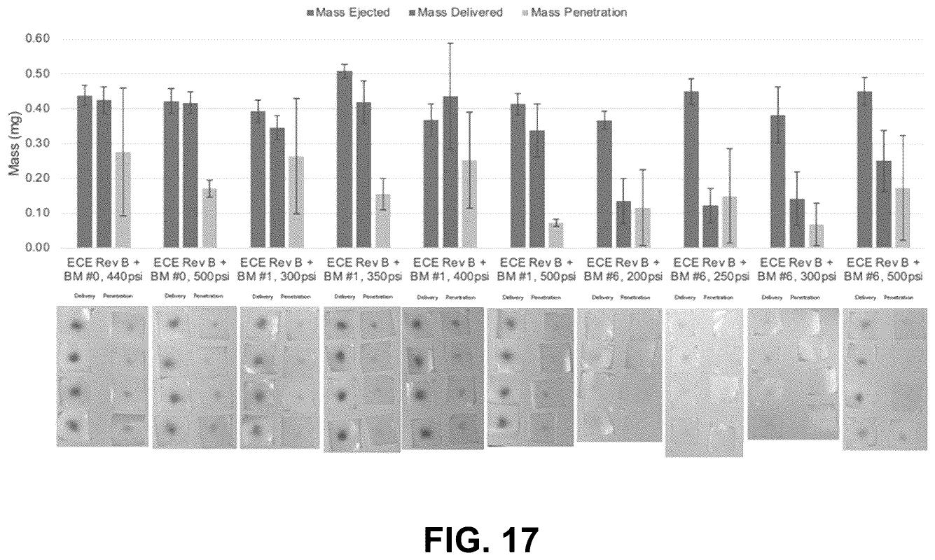

FIG. 17 is a graph illustrating the performance of burst membrane configurations operating at a range of driving pressures. In the displayed graph, burst membrane #0, burst membrane #1, and burst membrane #6 are operated at a range of burst pressures (e.g., ranging from 200 to 500 PSI) and evaluated based on an amount of mass ejected, an amount of mass delivered, and an amount of mass penetration. As illustrated, BM #0 requires the highest driving pressure to burst the membrane, BM #1 is intermediate at ˜250-250 PSI, and BM6 is lower at approximately 100 PSI. In one embodiment the device 1000 uses BM #1, which bursts consistently above a threshold to fully dislodge all particles at sufficient speed for full delivery and strong penetration but with a sufficiently low burst pressure that enables downsizing the solenoid valve and device to minimize effective driving or delivery pressure.

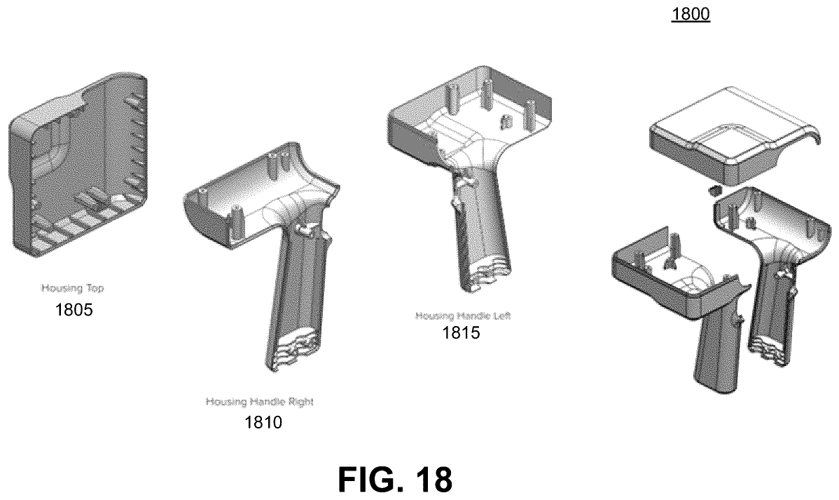

FIG. 18 illustrates a housing 1800 of the durable component 1010 of the device 1000. In the displayed embodiment, the housing 1800 is a three-piece enclosure comprising a housing top 1805, a right handle housing 1810, and a left handle housing 1815. The three-piece housing 1800 with integrated handle protects the internal subassembly of the device 1000 and supports a gas hose and power cable extending through the handle portion. In one embodiment, the housing components are manufactured using injection molding techniques and are comprised of Polycarbonate/Acrylonitrile Butadiene Styrene (PC/ABS) thermoplastics, though one of skill in the art will recognize that other manufacturing techniques and materials may be used.

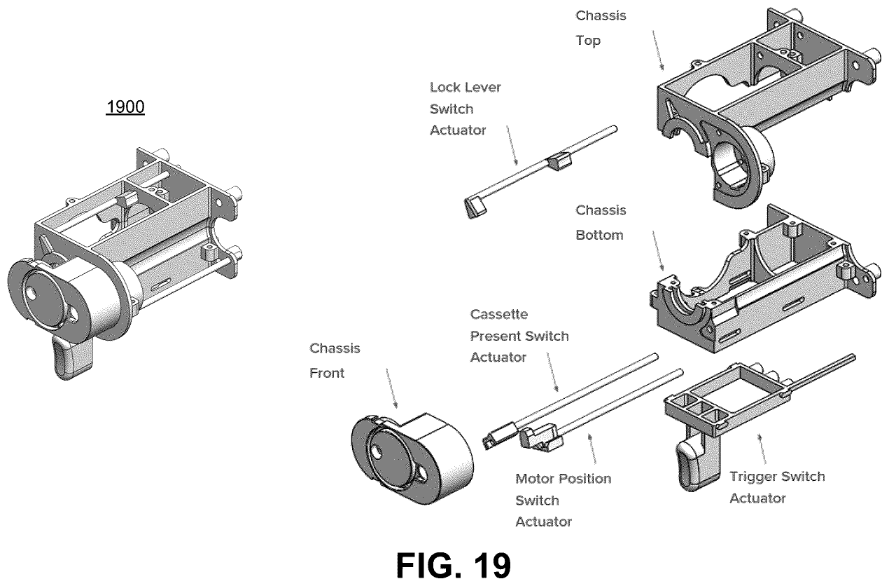

FIG. 19 illustrates an internal center chassis 1900 of the durable component 1010 of the device 1000. The chassis 1900 serves as a structural framework of the durable component 1000 to securely hold and organize the internal components including the solenoid, disposable locking mechanism, switch actuators, motor, and PCBA in designated positions within the housing 1800. For example, securing the solenoid within the chassis prevents vibration and misalignment, ensuring consistent and precise motion. Housing the locking mechanism within the chassis ensures that the locking mechanism aligns properly with disposable components and operates smoothly under pressure from the solenoid. The chassis further mechanically stabilizes the motor and PCBA to prevent stress, vibration, and accidental short circuit of the PCBA. In various embodiments, screws or other attachment mechanisms are used to secure the internal components in place within the chassis to minimize component shift or misalignment during operation and handling. The housing encloses or wraps around the solenoid/chassis assembly in a “floating” manner to ensure alignment and concentricity of gas path elements within the device 1000.

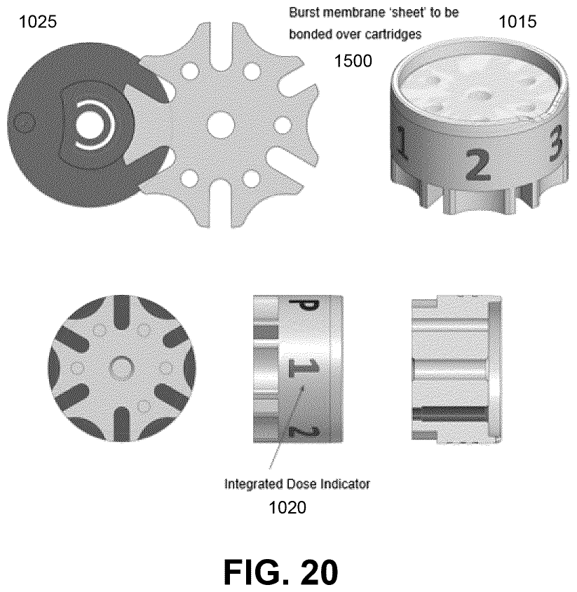

FIG. 20 illustrates the cassette 1015 of the device 1000, according to one embodiment. As discussed above with respect to FIGS. 11-14, in some embodiments, the device 1000 uses a Geneva wheel advancement mechanism for advancing the dose cylinder between the chambers housing doses of the biological or non-biological material. An integrated dose indicator 1020 on the exterior of the cassette 1015 indicates to the operator (e.g., through a window on the housing of the disposable component 1005) a dose number in the discharge position. In one embodiment, the Geneva wheel mechanism is configured to default to a purge (“P”) position and rotates between the chambers of the cassette 1015 as discussed above. In various configurations, the rotational positions are displayed in ascending (Dose 1, Dose 2, Dose 3, Dose 4, etc.) or descending (Dose 4, Dose 3, Dose 2, Dose 1) order from the purge position before reaching a final (e.g., “X”) position indicating to the operator that the cassette 1015 is empty and can no longer be fired. As discussed above, each motor revolution advances the Geneva wheel by one position, locking it securely in place.

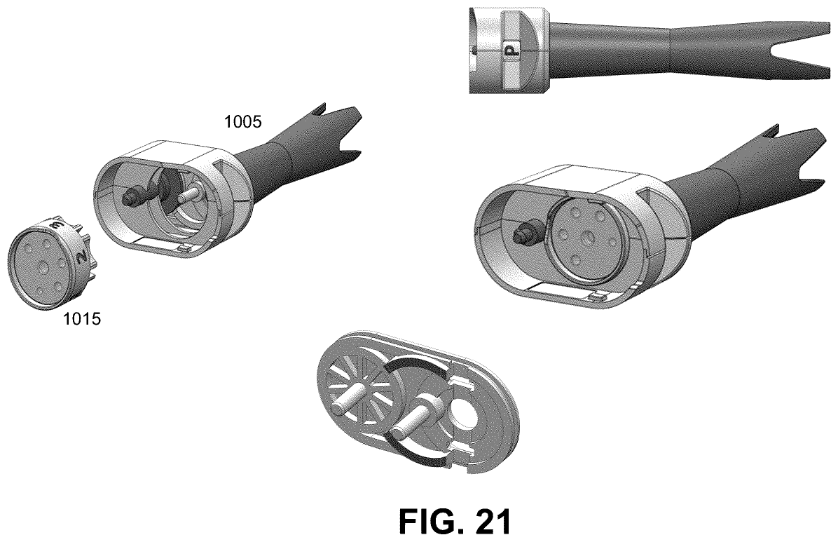

FIG. 21 illustrates installation of the cassette 1015 in the disposable component 1005 of the device 1000, according to one embodiment. To install the cassette 1015 into the disposable component 1005, the operator aligns the center pivot hole of the cassette 1015 with the disposable center pivot shaft. In one embodiment, the device 1000 provides visual and mechanical feedback indicating whether the Geneva wheel was correctly installed. For example, if the Geneva wheel is not inserted correctly, the device 1000 will not fire, and the green “ready” LED will remain off.

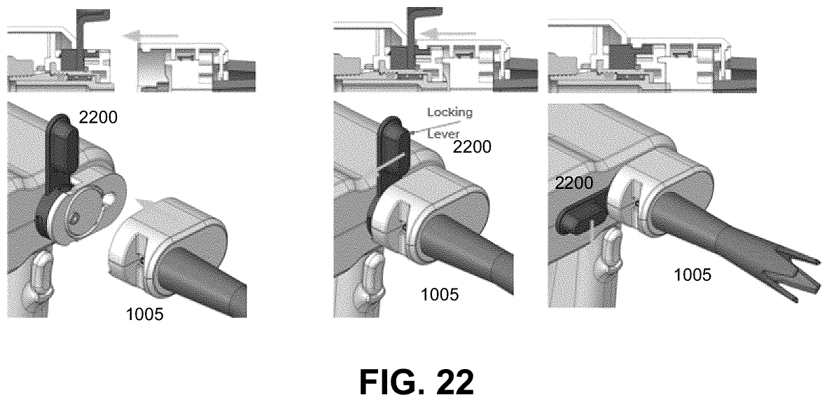

FIG. 22 illustrates a disposable attachment method using a locking lever, according to one embodiment. Once the cassette 1015 is installed, the operator attaches the disposable component 1005 to the durable component 1010 by placing the disposable component 1005 on the front of the durable component 1010 while a locking lever 2200 is in a vertical position. The operator rotates the lever 2200 (e.g., 90 degrees) to lock the disposable component 1005 in place, ensuring a secure and functional connection. Returning the lever 2200 to the vertical position causes the disposable component 1005 to detach from the durable component 1010.

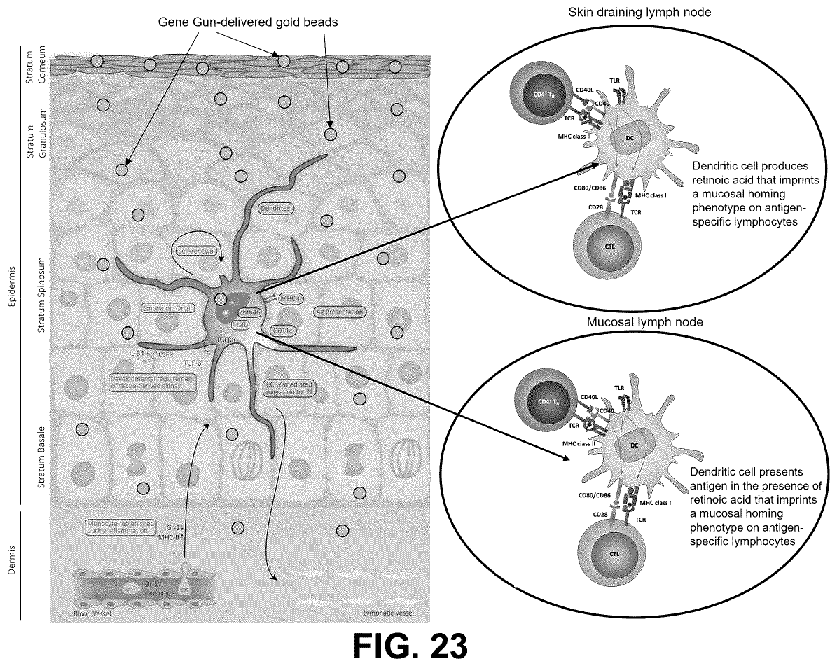

FIG. 23 illustrates improved clinical utility of the device 1000 over legacy devices. Vaccines delivered intramuscularly (systemically) elicit B and T lymphocyte immune responses that rest down to a memory state after an initial activated state. In this regard, B lymphocytes can differentiate into plasma cells that actively produce antibodies, or into memory B lymphocytes after antigen encounter. For many pathogens and systemically-delivered vaccines, elicited plasma cells can be short-lived while the memory B lymphocytes can persist long-term, but do not produce their antibodies until they are re-exposed to antigen. Similarly, systemic vaccination elicits T lymphocytes responses, but most expanded effector T lymphocytes die off and only a small pool of memory T lymphocytes is retained in circulation. These memory lymphocytes take less time to respond to antigen re-exposures than naïve lymphocytes. However, mounting an effective immune response after antigen re-exposure requires time since: an infection must establish before sufficient antigens are made to reactivate memory lymphocytes; antigen presenting dendritic cells must acquire pathogen antigens, then mature and traffic to secondary lymphoid tissues; the mature dendritic cells must present antigens to memory T and B lymphocytes; the memory B lymphocytes must convert into plasma cells and memory T lymphocytes must expand and convert into effector cells; plasma cells must secrete sufficient antibodies to combat the pathogens; activated effector T lymphocytes must traffic to the sites of infection, and if the infection is in a mucosal tissue, responding T lymphocytes must acquire a mucosal homing phenotype to enter mucosal sites. The time that it takes for this reactivation process to occur gives pathogens time to establish robust infections that can directly cause symptoms and can trigger innate immune responses and the killing of numerous infected host tissue cells that both can cause mild to severe symptoms.

Mucosal vaccination elicits classical systemic memory B and T lymphocyte responses like systemic vaccination but also elicits mucosal immunity (mucosal homing B and T lymphocytes). Mucosal immunity is distinct from systemic immunity and is highly desirable for vaccines. In this regard, mucosal infections and mucosal vaccinations elicit mucosal tissue resident B and T lymphocytes that retain an effector/memory phenotype meaning that: mucosal B and T lymphocytes are present at sites of first pathogen exposure/infection (i.e., they do not have to traffic from the blood), and mucosal B and T lymphocytes can rapidly enter an effector phase to quickly combat pathogens with antibodies and the killing of infected cells. For these reasons, mucosal immune responses can respond to infections much quicker than systemic immune responses and therefore can better blunt infections to dramatically reduce disease symptoms.

Mucosal homing lymphocytes and mucosal dendritic cells express the chemokine receptor CCR9, that is bound by the thymus-expressed chemokine TECK. TECK is constitutively expressed by cells of mucosal tissues, including epithelial cells and its constitutive expression attracts CCR9-expressing lymphocytes to mucosal immune effector sites such as the lamina propria regions of the gut and the respiratory tract. Mucosal homing lymphocytes and dendritic cells also express the integrin α4β7, which binds to the mucosal addressin cell adhesion molecule-1 (MAdCAM-1). MAdCAM-1 is expressed by high endothelial venules and allows α4β7 expressing cells to extravasate into mucosa-associated lymphoid tissues. Therefore, the co-expression of CCR9 and α4β7 differentiate mucosal homing lymphocytes and dendritic cells from their systemic counterparts and permit the segregation of systemic and mucosal compartments. The differences between the triggering of systemic and mucosal immune responses have been well-studied and it is known that retinoic acid is the master regulator of mucosal immunity. Briefly, if naïve lymphocytes are activated by antigen-bearing dendritic cells in the presence of retinoic acid, they will acquire a mucosal homing phenotype and home to mucosal sites. Dendritic cells seem to be the most important source of retinoic acid for imprinting a mucosal homing phenotype on the lymphocytes that they present antigens to.

Traditionally, it was thought that the delivery of vaccines to mucosal sites was the only way to evoke robust mucosal immunity since it appeared that only mucosal dendritic cells made retinoic acid. It was subsequently discovered that skin-resident dendritic cells, including epidermal Langerhans cells also make retinoic acid. This discovery helps explain why skin vaccination, and in particular epidermal vaccination, elicits better mucosal immune responses than intramuscular vaccination. Gene gun devices propel dried DNA or RNA vaccine-coated microparticles directly into cells of the epidermis and upper dermis, including keratinocytes and antigen presenting dermal dendritic cells and epidermal Langerhans cells. Gene gun vaccination therefore elicits both systemic and mucosal antibodies and T lymphocyte responses.

The sum of gas path geometries (in the durable and disposable components, pre and post cartridge), burst membrane, sealing measures, etc. enable the delivery of a higher percentage of overall payload to the epidermis (at 10 micron to 50-100 micron depth range) through the stratum corneum, minimizing a) the percentage of payload that is stuck ‘short’ in the stratum corneum, b) the percentage of payload goes too deep into the dermis beyond 100 microns, and c) an overconcentration of delivery in the center of the dose that can blunt gene expression and/or immunogenicity by necrosing recipient cells due to overload (physical overbombardment). The disclosed configuration therefore achieves both improved targeting to the epidermis and immune cells as well as a higher percentage of payload delivered.

### Additional Considerations

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments. This is done merely for convenience and to give a general sense of the disclosure. This description should be read to include one or at least one and the singular also includes the plural unless it is obvious that it is meant otherwise.

Upon reading this disclosure, those of skill in the art will appreciate still additional alternative structural and functional designs for a medical delivery device. Thus, while particular embodiments and applications have been illustrated and described, it is to be understood that the described subject matter is not limited to the precise construction and components disclosed herein and that various modifications, changes and variations which will be apparent to those skilled in the art may be made in the arrangement, operation and details of the method and apparatus disclosed. The scope of protection should be limited only by the following claims.

1. A medical delivery device comprising:
a disposable component comprising:
a cassette configured to carry a plurality of doses of biological or non-biological material, each dose positioned within a chamber of the cassette;
a membrane mounted on a first side of the cassette, the membrane configured to form a barrier between the material and an elongated barrel of the device;
a drive shaft mechanically coupled to the cassette on a second side of the cassette and configured to advance the cassette between chambers using a Geneva wheel mechanism; and
the elongated barrel having a body that expands from a first distal end to a second distal end, the first distal end adjacent to the cassette and the second distal end comprising a nozzle portion, the elongated barrel being shaped to allow a gas acceleration along the body to achieve at least a target gas velocity at the second distal end; and
a durable component comprising:
a motor configured to advance the drive shaft; and
a gas inlet configured to be connected to an external compressed gas source used to burst the membrane and propel a dose of the material to a subject through the elongated barrel.

2. The medical delivery device of claim 1, wherein the durable component further comprises a solenoid valve configured to control a gas flow between the gas inlet and a chamber of the cassette in a discharge position.

3. The medical delivery device of claim 2, wherein the solenoid valve is a DC-powered solenoid valve.

4. The medical delivery device of claim 1, wherein the durable component further comprises a plurality of sensors to prevent actuation of the solenoid valve when the disposable component is not correctly attached to the durable component.

5. The medical delivery device of claim 1, wherein the durable component further comprises one or more LED indicators configured to indicate a device status to an operator of the device.

6. The medical delivery device of claim 1, wherein the cassette comprises a wheel portion of the Geneva mechanism, the cassette configured to rotate between chambers responsive to movement of the drive shaft.

7. The medical delivery device of claim 1, wherein the disposable component is securely coupled to the durable component using a locking mechanism.

8. The medical delivery device of claim 1, further comprising a dose indicator positioned on an exterior of the cassette, the dose indicator viewable to the operator through a window in a housing of the disposable component.

9. The medical delivery device of claim 1, wherein the durable component further comprises a chassis providing structural support for the solenoid valve, switch actuators, the motor, and a printed circuit board assembly positioned within the durable component.

10. The medical delivery device of claim 1, wherein components of the durable assembly are enclosed within a plastic housing.

11. The medical delivery device of claim 1, wherein the membrane has a burst pressure of 200-500 pounds per square inch (PSI).

12. The medical delivery device of claim 1, wherein the device is operated at a driving pressure of 200-500 pounds per square inch (PSI).

13. A method for operating a medical delivery device, the method comprising:
activating the medical delivery device;
detecting attachment of a disposable component to a durable component of the medical delivery device, the disposable component comprising a multi-dose cassette containing a plurality of dose chambers, each chamber configured to carry a dose of biological or non-biological material for delivery to a subject;
verifying a starting position of the multi-dose cassette;
advancing the multi-dose cylinder to a first dose cassette using a Geneva wheel mechanism;
discharging the medical delivery device to deliver the dose contained in the first dose chamber through an elongated barrel in the disposable component to a subject;
advancing the multi-dose cassette to a subsequent dose chamber using the Geneva wheel mechanism; and
after discharge of each dose of material contained in a dose chamber of the multi-dose cassette, advancing the multi-dose cassette to a hard stop such that the medical delivery device cannot be discharged unless a replacement disposable component is attached to the durable component.

14. The method of claim 13, wherein the medical delivery device is configured to be connected to an external compressed gas source used to propel the material through the elongated barrel.

15. The method of claim 14, wherein a target gas velocity through the elongated barrel is at a supersonic velocity.

16. The method of claim 13, wherein the durable component comprises a solenoid valve configured to control a gas flow between the gas inlet and a chamber of the cassette in a discharge position.

17. The method of claim 13, wherein the cassette comprises a wheel portion of the Geneva mechanism, the cassette configured to advance between chambers responsive to movement of a drive shaft.

18. The method of claim 17, wherein advancement of the cassette causes a change of display of a dose indicator viewable to the operator through a window in a housing of the disposable component.

19. The method of claim 13, wherein the medical delivery device is activated responsive to a secure coupling of the disposable component to the durable component.

20. The method of claim 13, wherein the cassette includes a membrane mounted on a first side of the cassette, the membrane configured to form a barrier between the material and the elongated barrel and configured to burst responsive to application of at least a threshold amount of pressure.
